(19) United States
(12) Patent Application Publication
(10) Pub. No.: US 2004/0220270 A1
(43) Pub. Date: Nov. 4, 2004

(54) Methods and composition of treating glaucoma by modulating tyrosinase/L-DOPA pathway

(75) Inventors: Simon W. M. John, Bar Harbor, ME (US); Richard T. Libby, Bar Harbor, ME (US)

Correspondence Address: ROPES & GRAY LLP, ONE INTERNATIONAL PLACE, BOSTON, MA, 02110-2624, US

(73) Assignee: The Jackson Laboratory, Bar Harbor, ME

(21) Appl. No.: 10/793,604

(22) Filed: Mar. 4, 2004

Related U.S. Application Data
(60) Provisional application No. 60/452,805, filed on Mar. 7, 2003.

(51) Int. Cl. A61K 31/198

(52) U.S. Cl. 514/567

(57) ABSTRACT

In certain embodiments, the invention relates to methods of treating or preventing glaucoma. Such methods comprise administering to an individual a compound that increases DOPA or DOPA metabolite activities, and/or modulates at least one downstream signaling pathway. In other embodiments, the invention relates to methods of predicting or diagnosing glaucoma in an individual. Such methods comprise measuring the level of L-DOPA in the aqueous humor of the individual the individual, or measuring the function of a tyrosine hydroxylase or a tyrosinase in the individual. In yet other embodiments, the invention relates to novel pharmaceutical compositions for glaucoma therapy.

### CROSS-REFERENCE TO RELATED APPLICATIONS

The present application claims the benefit of U.S. Provisional Application No. 60/452,805, filed Mar. 7, 2003; the specification of which is hereby incorporated by reference in its entirety.

### FUNDING

Work described herein was funded, in whole or in part, by National Cancer Institute Grant CA34196. The United States government has certain rights in the invention.

### BACKGROUND OF THE INVENTION

Glaucoma is an ophthalmologic disorder responsible for visual impairment. It is one of the most common cause of blindness. The disease is characterized by a progressive neuropathy caused at least in part by deleterious effects resulting from increased intraocular pressure on the optic nerve. There are different types of glaucoma and often they have a multifactorial etiology (1-3). Glaucoma is a genetically heterogeneous cause of blindness that affects all age groups and all populations. Glaucoma may be either “primary” or “secondary.” Primary glaucoma results directly from anatomical and/or physiological disturbances in the flow of aqueous humor (e.g., intraocular fluid). Secondary glaucoma occurs as a sequel to ocular injury (e.g., trauma inflicted to the eye) or preexisting disease (e.g., an intraocular tumor or an enlarged cataract). Though the various secondary glaucomas have different etiologies, they are similar to the primary glaucoma in that they all produce visual loss through optic neuropathy.

The major types of primary glaucoma include: (i) open-angle glaucoma (also known as chronic or simple glaucoma); (ii) angle-closure glaucoma (also known as closed-angle or narrow-angle glaucoma); and (iii) congenital glaucoma (also known as infantile glaucoma). Open-angle glaucoma constitutes approximately 90% of all primary glaucoma, and is characterized by abnormally high resistance to fluid drainage from the eye. Angle-closure glaucoma entails closure or blockage of the anterior chamber angle by another ocular structure (usually the iris), and constitutes approximately 5% of primary glaucoma. Primary congenital glaucoma (PCG) is a small subset of glaucoma, but is severe and has a high incidence in some populations (1, 4). PCG results from poorly understood developmental abnormalities of the ocular drainage structures and is characterized by high intraocular pressure, corneal edema, photophobia and ocular enlargement (1, 3, 4). Recessive inheritance of PCG is common with almost complete penetrance in populations with a high consanguinity rate. There is often reduced penetrance (40% in some populations) and variable presentation, however, pointing to a multifactorial etiology (OMIM 231300) (4-6). Although there is evidence for a modifier gene affecting the development of glaucoma in individuals homozygous for CYP1B1 mutations, the gene(s) has not yet been identified (7).

As described above, untreated glaucoma can result in severe consequences, including blindness. Clearly, there is a need for new and additional approaches for treating conditions such as glaucoma, which are significant public health problem.

### SUMMARY OF THE INVENTION

The present invention relates to methods of treating or preventing glaucoma through administrating a compound that increases DOPA and/or DOPA metabolite activities, and/or modulates a (at least one; one or more) downstream signaling pathway. In specific embodiments, the method comprises administering a compound that increases L-DOPA and/or L-DOPA metabolite activities, and/or modulates a downstream signaling pathway.

In certain embodiments, the invention provides methods of treating an individual suffering from glaucoma. An individual (patient or subject) suffering from glaucoma is treated by administering a therapeutically effective amount of a compound that increases DOPA and/or DOPA metabolite activities, and/or modulates a (at least one; one or more) downstream signaling pathway. In one embodiment, the compound that is administered is selected from the group consisting of: L-DOPA, dopaquinone, dopamine, an L-DOPA derivative, an L-DOPA metabolite, and a catecholamine. In another embodiment, the compound that is administered is one which increases the activity of an enzyme (at least one; one or more) with tyrosine hydroxylase activity or tyrosine monooxygenase activity, for example, by increasing the activity or expression level of such an enzyme. The onset of the glaucoma can be either in the juvenile or in the adult. Optionally, the compounds employed in these methods can be formulated with a pharmaceutically acceptable carrier. In certain embodiments, the compounds are administered orally, intravitreally, topically, ocularly or parenterally. In certain embodiments, these methods of treatment further comprise administering a second glaucoma therapeutic agent selected from the group consisting of: a miotic, epinephrine; a beta-blocker; a carbonic anhydrase inhibitor; an alpha-adrenergic agonist; and a prostaglandin analog. These methods can further comprise treating an individual with a glaucoma therapy selected from the group consisting of: trabeculoplasty; iridotomy; and cyclophotocoagulation.

The present invention also relates to methods of reducing damage to retinal ganglion cells associated with glaucoma in an individual. An individual (patient or subject) suffering from glaucoma is treated by administering a therapeutically effective amount of a compound that increases DOPA and/or DOPA metabolite activities, and/or modulates a (at least one; one or more) downstream signaling pathway, in an amount sufficient to reduce glaucoma-associated damage to the cells. In one embodiment, the compound that is administered is selected from the group consisting of: L-DOPA, dopaquinone, dopamine, an L-DOPA derivative, an L-DOPA metabolite, and a catecholamine. In another embodiment, the compound that is administered is one which increases the activity of an enzyme (at least one; one or more) with tyrosine hydroxylase activity or tyrosine monooxygenase activity, for example, by increasing the activity or expression level of such an enzyme. Here, too, the onset of the glaucoma can be either in the juvenile or in the adult. Optionally, the compounds employed in these methods can be formulated with a pharmaceutically acceptable carrier. In certain embodiments, the compounds are administered orally, intravitreally, topically, ocularly or parenterally. In certain embodiments, these methods further comprise administering a second glaucoma therapeutic agent selected from the group consisting of: a miotic, epinephrine; a beta-blocker; a carbonic anhydrase inhibitor; an alpha-adrenergic agonist; and a prostaglandin analog. These methods can further comprise treating an individual with a glaucoma therapy selected from the group consisting of: trabeculoplasty; iridotomy; and cyclophotocoagulation. Any combination of therapies for glaucoma can be used.

In certain embodiments, the invention provides methods of preventing or reducing (delaying the timing of or reducing the severity of) the onset of glaucoma in an individual. An individual (patient or subject) is administered a therapeutically effective amount of a compound that increases DOPA and/or DOPA metabolite activities, and/or modulates a (at least one; one or more) downstream signaling pathway. In one embodiment, the compound is selected from the group consisting of: L-DOPA, dopaquinone, dopamine, an L-DOPA derivative, an L-DOPA metabolite, and a catecholamine. In another embodiment, the compound that is administered is one which increases the activity of an enzyme (at least one; one or more) with tyrosine hydroxylase activity or tyrosine monooxygenase activity, for example, by increasing the activity or expression level of such an enzyme. The onset of the glaucoma can be either in the juvenile or in the adult. Optionally, the compounds employed in these methods can be formulated with a pharmaceutically acceptable carrier. In certain embodiments, the compounds are administered orally, intravitreally, topically, ocularly or parenterally.

In certain embodiments, the invention provides methods of predicting glaucoma in an individual. In one embodiment, the methods comprise measuring the level of L-DOPA in the aqueous humor of the individual. A reduced level of L-DOPA in the aqueous humor is indicative of increased likelihood that the individual will develop glaucoma. In another embodiment, the methods comprise measuring the function (e.g., activity and/or expression level) of an enzyme (at least one; one or more) with tyrosine hydroxylase activity or tyrosine monooxygenase activity. A reduced function of the enzyme is indicative of increased likelihood that the individual will develop glaucoma.

In certain embodiments, the invention provides methods of diagnosing glaucoma in an individual. In one embodiment, the methods comprise measuring the level of L-DOPA in the aqueous humor of the individual. A reduced level of L-DOPA in the aqueous humor is indicative of glaucoma in the individual. In another embodiment, the methods comprise measuring the function (e.g., activity and expression level) of an enzyme (at least one; one or more) with tyrosine hydroxylase activity or tyrosine monooxygenase activity in the individual. A reduced function of the enzyme is indicative of glaucoma in the individual.

In certain embodiments, the invention provides methods of treating an individual with ocular manifestation associated with albinism or with ocular albinism. Such methods comprise administering to the individual a therapeutically effective amount of a compound that increases DOPA and/or DOPA metabolite activities, and/or modulates a downstream signaling pathway. In one embodiment, the compound is selected from the group consisting of: L-DOPA, dopaquinone, dopamine, an L-DOPA derivative, an L-DOPA metabolite, and a catecholamine. In another embodiment, the compound increases the mono-oxygenation or hydroxylation of tyrosine, for example, by increasing the activity or expression level of an enzyme (at least one; one or more) with tyrosine hydroxylase activity or tyrosine monooxygenase activity.

In certain embodiments, the invention provides pharmaceutical compositions which comprise: (a) a first compound that increases DOPA and/or DOPA metabolite activities, and/or modulates a (at least one; one or more) downstream signaling pathway, and (b) a second compound selected from the group consisting of: a miotic, epinephrine; a beta-blocker; a carbonic anhydrase inhibitor; an alpha-adrenergic agonist; and a prostaglandin analog. In one embodiment, the compound is selected from the group consisting of: L-DOPA, dopaquinone, dopamine, an L-DOPA derivative, an L-DOPA metabolite, and a catecholamine. In another embodiment, the compound increases the mono-oxygenation or hydroxylation of tyrosine, for example, by increasing the activity or expression level of an enzyme (at least one; one or more) with tyrosine hydroxylase activity or tyrosine monooxygenase activity.

### BRIEF DESCRIPTION OF THE DRAWINGS

The file of this patent contains at least one drawing executed in color. Copies of this patent with color drawing(s) will be provided by the Patent and Trademark Office upon request and payment of the necessary fee.

FIGS. 1A-1F show ocular abnormalities in Cyp1b1^{-/-} mice. All images are from adult mice of the 129X1/SvJX B6 mixed background. (A) In Cyp1b1^{+/+} mice, the iridocorneal angle is well formed with an obvious Schlemm's canal (SC) of normal extent. The trabecular meshwork (TM) and iris (I) are normal and the deep angle recess of the anterior chamber (AC) is open. (B) Pigmented Cyp1b1^{-/-}. The trabecular meshwork is hypoplastic and material resembling Descemet's membrane (basal lamina of the corneal endothelium, arrowheads) abnormally covers a portion of the trabecular meshwork. (C) Albino Cyp1b1^{-/-}. A very small Schlemm's canal may be present (arrowhead). The trabecular meshwork cannot be identified. The deep angle recess is not open due to a synechia (*). (D) Cyp1b1^{+/+}. There is an endothelial lined Schlemm's canal and a robust trabecular meshwork with numerous well-formed trabecular beams. The internal aspect of the trabecular meshwork is demarcated by the anterior chamber. (E) Cyp1b1^{-/-}. The endothelial lining of Schlemm's canal is attenuated and there is only a single trabecular beam. Access to the trabecular meshwork and Schlemm's canal is blocked by a thick basal lamina (BL). The inner aspect of the basal lamina is covered with abnormal cells resembling endothelial cells (E). (F) Cyp1b1^{-/-}. Schlemm's canal is lined by endothelium and contains a giant vacuole (aqueous humor drainage structure, V). Only 1-2 poorly formed trabecular beams are present. Smooth muscle (SM) is abnormally located in the trabecular meshwork. Inset: Higher magnification demonstrates focally increased density of the plasma membrane (arrow), pinocytotic vesicles (black arrowhead), and longitudinally arranged actin filaments (white arrowhead), confirming the presence of smooth muscle. Scale bars: (A to C), 40 μm; (D to F), 1 μm.

FIGS. 2A-2F show that tyrosinase modifies angle defects in Cyp1b1^{-/-} mice. All mice were adult with a B6 genetic background. Common features are shown for mice of each genotype. (A) Cyp1b1^{+/+} Tyr^{+/+} (pigmented). There is a well-formed angle with a long open Schlemm's canal (SC) and robust trabecular meshwork (TM). (B) Cyp1b1^{-/-} Tyr^{+/+}. Schlemm's canal and the trabecular meshwork are present. This is true around most of the eye. (C) Cyp1b1^{+/+} Tyr^{c-2J/c-2J} (albino). Schlemm's canal and the trabecular meshwork are well formed. (D) Cyp1b1^{-/-} Tyr^{C-2J/c-2J}. Schlemm's canal and trabecular meshwork are not identifiable. The iris is attached by a synechia (*) to the region that is normally occupied by trabecular meshwork. (E) Mean severity grades demonstrate that tyrosinase activity modifies the severity of angle abnormalities in Cyp1b1^{-/-} mice that are genetically uniform except for their Tyr genotypes. For an eye, the grade can be any value between 0 and 144 (12). A grade of 0 would indicate that the angle was completely normal at all analyzed locations around the eye, while a score of 144 would indicate that the angle was severely affected at all locations studied. Angles with severe and extensive developmental lesions have high grades while angles with less severe or less extensive lesions have lower grades. Severity was assessed for two important structures that are known to be affected by anterior segment dysgenesis in humans, Schlemm's canal (SC) and the trabecular meshwork (TM). Also, a grade was assigned for the presence and extent of a synechia (Syn). Cyp^{-/-} Tyr^{-2J/c-2J} mice had significantly more extensive dysgenesis than Cyp^{-/-} Tyr^{+/+} mice (p<0.001 for all phenotypes assessed). F. Mean severity grades demonstrated that Tyr genotype affects angle development in otherwise wild type, genetically identical B6 mice. Cyp1b1^{+/+} Tyr^{c-2J/c-2J} mice had focal developmental defects whereas Cyp1b1^{+/+} Tyr^{+/+} mice had none (P<0.001 for all phenotypes assessed). Pigmented and albino mice of each genotype were intermixed during analysis and the grader was unaware of the genotypes. Scale bars, 40 μm.

FIGS. 3A-3E s how that L-DOPA treatment alleviates the effect of angle dysgenesis. All mice were Cyp1b1^{-/-} Tyr^{c-2J/c-2J} with a B6 genetic background. (A) In an untreated mouse there is a severely malformed iridocomeal angle with a large synechia (*) attaching the iris (I) and the cornea (C). There is no observable Schlemm's canal (SC) or trabecular meshwork (TM). Schlemm's canal and the trabecular meshwork normally extend to the arrowhead. (B) LDOPA supplementation (1 mg/ml in drinking water) substantially prevented developmental abnormalities in the iridocomeal angle. In this representative section, there is a long endothelial lined Schlemm's canal, the trabecular meshwork is robust, and the angle recess is open. Electron micrographs of untreated (C) and L-DOPA treated (D) mice show that L-DOPA treatment rescues the ultrastructure of the trabecular meshwork. (C) In an untreated mouse Schlemm's canal is completely absent, the trabecular meshwork is severely hypoplastic and contains little extracellular matrix, and the trabecular meshwork is attached to the cornea. (D) In a L-DOPA treated mouse (at a similar location to that shown for the untreated mouse in C), Schlemm's canal is present with a normal endothelial lining and the trabecular meshwork is robust with organized trabecular beams. (E) Mean severity grades demonstrate that the developmental dysgenesis in Cyp1b1^{-/-} Tyr^{c-2J/c-2J} double mutants can be alleviated by L-DOPA supplementation (SC, Schlemm's canal; TM, Trabecular mechwork;

Syn, synechiae; p<0.001 for all assessed phenotypes). The grader was not aware of which mice were from which treatment arm. Scale bars: (A and B), 40 μm; (C and D), 1 μm.

FIGS. 4A-4C show that grossly, the anterior segments of Cyp1b1^{+/+} and Cyp1b1^{-/-} mice have normal morphology with complex iris detail and small round pupils. (C) Albino Cyp1b1^{-/-}. Indicating the variability of abnormalities seen in these mice, Schlemm's c anal and the trabecular meshwork are relatively normal in this region of the same eye shown in FIG. 1C (same section but opposite angle). Scale bar: (C) 40 μm.

FIGS. 5A-5C show that tyrosinase modifies angle defects in Foxc1 mutant mice. All mice are adult Foxc1^{+/-} on a B6 genetic background. Due to phenotypic variability, common features are shown for mice of each genotype. (A) Foxc1^{+/-Tyr+/+} (pigmented). Schlemm's canal (SC) and the iris (I) are normal. The trabecular meshwork (arrowheads) is mildly hypoplastic. (B) Foxc1^{+/-} Tyr^{c-2J/c-2J} (albino). Schlemm's canal and the trabecular meshwork cannot be identified. A broad synechiae (asterisk) occupies the region where the trabecular meshwork is normally located. (C) Foxc1^{+/-} Tyr^{c-2J/c-2J} (albino) mice had more severe angle dysgenesis than Foxc1^{+/ -} Tyr^{+/+} (pigmented) mice that were otherwise genetically matched (p<0.001 for all phenotypes). This demonstrates that Tyr mutation exacerbates angle dysgenesis in Foxc1^{+/-} eyes. Slides from Foxc1^{+/-} mice were intermixed with slides from appropriate Foxc1^{+/+} mice during grading, so that the grader was unaware of the genotypes. Scale bars, 40 μm.

FIG. 6 shows the tyrosine hydroxylase step in the synthetic pathway of L-DOPA and dopamine.

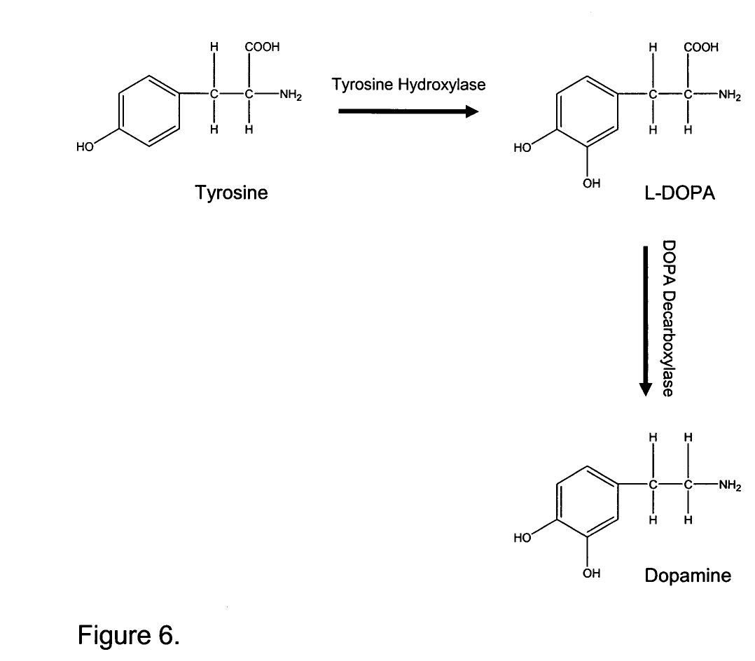

### DETAILED DESCRIPTION OF THE INVENTION

The current invention is based, in part, on Applicants' discovery that signaling through tyrosinase/L-DOPA pathway contributes to glaucoma development. Applicants identified the tyrosinase gene (Tyr) as a modifier of the drainage structure phenotype, with Tyr deficiency increasing the magnitude of dysgenesis. The severe dysgenesis in eyes was alleviated by administration of the tyrosinase product L-DOPA (dihydroxyphenylalanine).

In certain embodiments, the invention provides methods of treating an individual suffering from glaucoma. Glaucoma is an ophthalmologic disorder typically characterized by an increase in intraocular pressure, atrophy of retinal ganglion cells of the optic nerve or both, which often results in impaired vision. It is characterized by a progressive neuropathy caused at least in part by deleterious effects resulting from intraocular pressure on the optic nerve. The term glaucoma refers broadly to both primary glaucoma, which includes open-angle, angle-closure, and congenital glaucoma, and secondary glaucoma, which occur as a sequel to ocular injury or preexisting disease. The present methods can be used to treat glaucoma in an individual of any age (e.g., infant, child, juvenile, adult) and of any etiology.

In these embodiment, an individual (patient or subject) suffering from glaucoma is treated by administering a therapeutically effective amount of a compound that increases DOPA and/or DOPA metabolite activities, and/or modulates a (at least one; one or more) downstream signaling pathway. In one embodiment, the compound that is administered is selected from the group consisting of: L-DOPA, dopaquinone, dopamine, an L-DOPA derivative, an L-DOPA metabolite, and a catecholamine. In another embodiment, the compound that is administered is one which increases the activity of an enzyme (at least one; one or more) with tyrosine hydroxylase activity or tyrosine monooxygenase activity, for example, by increasing the activity or expression level of such an enzyme. The onset of the glaucoma can be either in the juvenile or in the adult. Optionally, the compounds employed in these methods can be formulated with a pharmaceutically acceptable carrier. In certain embodiments, the compounds are administered orally, intravitreally, topically, ocularly or parenterally. In certain embodiments, these methods of treatment further comprise administering a second glaucoma therapeutic agent selected from the group consisting of: a miotic, epinephrine; a beta-blocker; a carbonic anhydrase inhibitor; an alpha-adrenergic agonist; and a prostaglandin analog. These methods can further comprise treating an individual with a glaucoma therapy selected from the group consisting of: trabeculoplasty; iridotomy; and cyclophotocoagulation. Any combination of therapies for glaucoma can be used.

The present invention also relates to methods of reducing damage to retinal ganglion cells associated with glaucoma in an individual. An individual (patient or subject) suffering from glaucoma is treated by administering a therapeutically effective amount of a compound that increases DOPA and/or DOPA metabolite activities, and/or modulates a (at least one; one or more) downstream signaling pathway, in an amount sufficient to reduce glaucoma-associated damage to the cells. In one embodiment, the compound that is administered is selected from the group consisting of: L-DOPA, dopaquinone, dopamine, an L-DOPA derivative, an L-DOPA metabolite, and a catecholamine. In another embodiment, the compound that is administered is one which increases the activity of an enzyme (at least one; one or more) with tyrosine hydroxylase activity or tyrosine monooxygenase activity, for example, by increasing the activity or expression level of such an enzyme. Here, too, the onset of the glaucoma can be either in the juvenile or in the adult. Optionally, the compounds employed in these methods can be formulated with a pharmaceutically acceptable carrier. In certain embodiments, the compounds are administered orally, intravitreally, topically, ocularly or parenterally. In certain embodiments, these methods further comprise administering a second glaucoma therapeutic agent selected from the group consisting of: a miotic, epinephrine; a beta-blocker; a carbonic anhydrase inhibitor; an alpha-adrenergic agonist; and a prostaglandin analog. These methods can further comprise treating an individual with a glaucoma therapy selected from the group consisting of: trabeculoplasty; iridotomy; and cyclophotocoagulation.

In certain embodiments, the invention provides methods of preventing or reducing (delaying the timing of or reducing the severity of) the onset of glaucoma in an individual. For example, an individual who is at risk of developing glaucoma (e.g., an individual whose family history includes glaucoma) and/or has signs he/she will develop glaucoma can be treated by the present methods. In these embodiment, an individual (patient or subject) is administered a therapeutically effective amount of a compound that increases DOPA and/or DOPA metabolite activities, and/or modulates a (at least one; one or more) downstream signaling pathway. In one embodiment, the compound is selected from the group consisting of: L-DOPA, dopaquinone, dopamine, an L-DOPA derivative, an L-DOPA metabolite, and a catecholamine. In another embodiment, the compound that is administered is one which increases the activity of an enzyme (at least one; one or more) with tyrosine hydroxylase activity or tyrosine monooxygenase activity, for example, by increasing the activity or expression level of such an enzyme. The onset of the glaucoma can be either in the juvenile or in the adult. Optionally, the compounds employed in these methods can be formulated with a pharmaceutically acceptable carrier. In certain embodiments, the compounds are administered orally, intravitreally, topically, ocularly or parenterally.

In certain embodiments, the invention provides methods of predicting glaucoma in an individual. In one embodiment, the methods comprise measuring the level of L-DOPA in the aqueous humor of the individual. A reduced level of L-DOPA in the aqueous humor is indicative of increased likelihood that the individual will develop glaucoma. In another embodiment, the methods comprise measuring the function (e.g., activity and/or expression level) of an enzyme (at least one; one or more) with tyrosine hydroxylase activity or tyrosine monooxygenase activity. A reduced function of the enzyme is indicative of increased likelihood that the individual will develop glaucoma.

In certain embodiments, the invention provides methods of diagnosing glaucoma in an individual. In one embodiment, the methods comprise measuring the level of L-DOPA in the aqueous humor of the individual. A reduced level of L-DOPA in the aqueous humor is indicative of glaucoma in the individual. In another embodiment, the methods comprise measuring the function (e.g., activity and expression level) of an enzyme (at least one; one or more) with tyrosine hydroxylase activity or tyrosine monooxygenase activity in the individual. A reduced function of the enzyme is indicative of glaucoma in the individual.

In certain embodiments, the invention provides methods of treating an individual with ocular manifestation associated with albinism or with ocular albinism. Such methods comprise administering to the individual a therapeutically effective amount of a compound that increases DOPA and/or DOPA metabolite activities, and/or modulates a downstream signaling pathway. In one embodiment, the compound is selected from the group consisting of: L-DOPA, dopaquinone, dopamine, an L-DOPA derivative, an L-DOPA metabolite, and a catecholamine. In another embodiment, the compound increases the mono-oxygenation or hydroxylation of tyrosine, for example, by increasing the activity or expression level of an enzyme (at least one; one or more) with tyrosine hydroxylase activity or tyrosine monooxygenase activity.

In certain embodiments, the invention provides pharmaceutical compositions which comprise: (a) a first compound that increases DOPA and/or DOPA metabolite activities, and/or modulates a (at least one; one or more) downstream signaling pathway, and (b) a second compound selected from the group consisting of: a miotic, epinephrine; a beta-blocker; a carbonic anhydrase inhibitor; an alpha-adrenergic agonist; and a prostaglandin analog. In one embodiment, the compound is selected from the group consisting of: L-DOPA, dopaquinone, dopamine, an L-DOPA derivative, an L-DOPA metabolite, and a catecholamine. In another embodiment, the compound increases the mono-oxygenation or hydroxylation of tyrosine, for example, by increasing the activity or expression level of an enzyme (at least one; one or more) with tyrosine hydroxylase activity or tyrosine monooxygenase activity.

Therapeutic Compounds for Glaucoma

In certain embodiments, the present invention contemplates therapeutic compounds for use in treating glaucoma. Such compounds increase DOPA and/or DOPA metabolite activities, and/or modulate a (at least one; one or more) downstream signaling pathway. It is generally believed that the biosynthesis of dopamine (DA) in catecholaminergic neurons is regulated by a tyrosine hydroxylase, which converts tyrosine into L-DOPA. In melanocytes, a tyrosinase catalyzes both the hydroxylation of tyrosine and the consequent oxidation of L-DOPA to form melanin.

In one embodiment, the therapeutic compound is selected from the group consisting of: L-DOPA, dopaquinone, dopamine, an L-DOPA derivative, an L-DOPA metabolite, and a catecholamine. L-DOPA (3,4-dihydroxyphenylalanine) has been developed as a precursor of dopamine to be used to compensate for deficiency in dopamine in the brain of patients with Parkinson's disease, and is now generally accepted as the first drug of choice in the field. Various L-DOPA derivatives (e.g., prodrugs) have been developed, including salts and esters of L-DOPA. Exemplary L-DOPA derivatives and methods of preparing these L-DOPA derivatives can be found in U.S. Pat. Nos: 4,966,915; 5,354,885; 5,607,969; and 5,840,756.

In another embodiment, the compounds increase the mono-oxygenation or hydroxylation of tyrosine, for example, by increasing the activity or expression level of an enzyme with tyrosine hydroxylase activity or tyrosine monooxygenase activity. These compounds include a protein, peptide, small organic molecule, nucleic acid, peptidomimetic, soluble chemokine receptor, and antibody. The stimulatory effect of such compounds may result from either directly acting on an enzyme with tyrosine hydroxylase activity or tyrosine monooxygenase activity or indirectly acting on an inhibitor of such enzyme.

Potential compounds may include a small molecule (such as a peptidomimetic) that binds to an enzyme with tyrosine hydroxylase activity or tyrosine monooxygenase activity, making it either more readily accessible or inaccessible to the other binding partner such that normal biological activity is enhanced. Examples of small molecules include, but are not limited to, small peptides or peptide-like molecules (e.g., a peptidomimetic). As used herein, the term “peptidomimetic” includes chemically modified peptides and peptide-like molecules that contain non-naturally occurring amino acids, peptoids, and the like. Peptidomimetics provide various advantages over a peptide, including enhanced stability when administered to a subject. Methods for identifying a peptidomimetic are well known in the art and include the screening of databases that contain libraries of potential peptidomimetics.

In certain embodiments, such therapeutic compounds also encompass numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl, sulfhydryl or carboxyl group.

Candidate compounds can be obtained from a wide variety of sources, including libraries of synthetic or natural compounds. For example, numerous means are available for random and directed synthesis of a wide variety of organic compounds and biomolecules, including expression of randomized oligonucleotides. Alternatively, libraries of natural compounds in the form of bacterial, fungal, plant, and animal extracts are available or readily produced. Additionally, natural or synthetically produced libraries and compounds can be modified through conventional chemical, physical, and biochemical means. Known pharmacological agents may be subjected to directed or random chemical modifications, such as acylation, alkylation, esterification, and amidification, to produce structural analogs.

The present invention also contemplates therapeutic compounds that are obtainable from the screening methods described as below.

Screening Assays

Methods of the present invention also employ therapeutic compounds which can be identified by a variety of screening methods. In certain embodiment, such compounds stimulate functions (e.g., activity or expression level) of an enzyme with tyrosine hydroxylase activity or tyrosine monooxygenase activity. The therapeutic compounds can be employed for therapeutic and prophylactic purposes for glaucoma.

In certain embodiments, such screening procedures involve providing appropriate cells that express an enzyme which increases mono-oxygenation or hydroxylation of tyrosine (e.g., an enzyme with tyrosine hydroxylase activity or tyrosine monooxygenase activity). Such cells include cells from mammals, yeast, Drosophila, and E. coli. In particular, a polynucleotide encoding such an enzyme is employed to transfect cells to thereby express the enzyme. Cells expressing the enzyme are contacted with a test compound (agent) to determine its ability to increase DOPA and/or DOPA metabolite activities (e.g., L-DOPA or L-DOPA metabolite activities), modulate a downstream signaling pathway, or increase synthesis of DOPA and/or DOPA metabolites (e.g., L-DOPA or L-DOPA metabolites).

Other screening assays that detect the expression level (protein or nucleic acid) of an enzyme with tyrosine hydroxylase activity or tyrosine monooxygenase activity, may be used for screening for therapeutic compounds. Methods of detecting and optionally quantitating proteins can be achieved by techniques such as antibody- based detection assays. In these cases, antibodies may be used in a variety of detection techniques, including enzyme-linked immunosorbent assays (ELISAs), immunoprecipitations, and Western blots. On the other hand, methods of detecting and optionally quantitating nucleic acids generally involve preparing purified nucleic acids and subjecting the nucleic acids to a direct detection assay or an amplification process followed by a detection assay. Amplification may be achieved, for example, by polymerase chain reaction (PCR), reverse transcriptase (RT), and coupled RT-PCR. Detection of nucleic acids is generally accomplished by probing the purified nucleic acids with a probe that hybridizes to the nucleic acids of interest, and in many instances, detection involves an amplification as well. Northern blots, dot blots, microarrays, quantitative PCR, and quantitative RT-PCR are all well known methods for detecting nucleic acids.

Methods of Treatment

In certain embodiments, the present invention provides methods of treating an individual suffering from glaucoma. In other embodiments, the invention provides methods of preventing or reducing the onset of glaucoma in an individual. For example, an individual who is at risk of developing glaucoma (e.g., an individual whose family history includes glaucoma) and/or has signs he/she will develop glaucoma can be treated by the present methods. These methods comprise administering to the individual an effective amount of a compound that increases DOPA and/or DOPA metabolite activities, and/or modulates a downstream signaling pathway. These methods are particularly aimed at therapeutic and prophylactic treatments of animals, and more particularly, humans.

In certain embodiments of such methods, one or more compounds can be administered, together (simultaneously) or at different times (sequentially). In addition, such compounds can be administered with another type(s) of compound(s) for treating glaucoma such as a miotic, epinephrine, a beta-blocker, a carbonic anhydrase inhibitor, an alpha-adrenergic agonist or a prostaglandin analog. The two types of compounds may be administered simultaneously or sequentially.

In other embodiments, these methods are further combined with another type of glaucoma therapy. Exemplary glaucoma therapies include, but are not limited to, trabeculoplasty; iridotomy; and cyclophotocoagulation. Any combination of therapies for glaucoma can be used.

In certain embodiments, gene therapy may be applicable with the use of nucleic acids. For example, a nucleic acid encoding an enzyme with tyrosine hydroxylase or tyrosine monooxygenase can be employed to increase L-DOPA production and possibly activate a (at least one; one or more) downstream signaling pathway. Alternatively, an antisense nucleic acid which reduce or inhibit expression of an inhibitor of such an enzyme can be used for the same purpose.

Formulation and Administration

In certain embodiments of the present invention, therapeutic compounds for glaucoma (e.g., L-DOPA, dopaquinone, dopamine, an L-DOPA derivative, an L-DOPA metabolite, and a catecholamine) may be formulated in combination with a suitable pharmaceutical carrier. Such formulations comprise a therapeutically effective amount of the compound, and a pharmaceutically acceptable carrier (excipient). Examples of suitable carriers are well known in the art. To illustrate, the pharmaceutically acceptable carrier can be an aqueous solution or physiologically acceptable buffer. Optionally, the aqueous solution is an acid buffered solution. Such acid buffered solution may comprise hydrochloric, sulfuric, tartaric, phosphoric, ascorbic, citric, fumaric, maleic, or acetic acid. Alternatively, such carriers include, but are not limited to, saline, buffered saline, dextrose, water, glycerol, ethanol, and combinations thereof. Formulations will suit the mode of administration, and are well within the skill of the art.

The phrase “therapeutically effective amount,” as used herein, refers to an amount that is sufficient or effective to prevent or treat (prevent the progression of or reverse) glaucoma, including alleviating symptoms of glaucoma. For example, a therapeutically effective amount of a compound decreases elevated intraocular pressure and/or reduces glaucoma associated damage.

The subject compounds may be employed alone or in conjunction with another type of compound for treating glaucoma such as a miotic, epinephrine, a beta-blocker, a carbonic anhydrase inhibitor, an alpha-adrenergic agonist or a prostaglandin analog. These different types of compounds may be administered in the same formulation or in a separate formulation.

Forms of systemic administration of the pharmaceutical compositions include injection, typically by intravenous injection. Other injection routes, such as subcutaneous, intramuscular, or intraperitoneal, can be used. Alternative means for systemic administration include transmucosal and transdermal administration using penetrants such as bile salts or fusidic acids or other detergents. In addition, oral administration may also be possible if the compounds are properly formulated in enteric or encapsulated formulations. Administration of these compounds may also be topical, intravitreal, ocular or perenteral. A preferred mode of administration for these compounds may be in the form of a solution or suspension that can be administered as an eye drop.

The dosage range depends on the choice of the compound, the route of administration, the nature of the formulation, the nature of the subject's condition, and the judgment of the attending practitioner. Wide variations in the needed dosage, however, are to be expected in view of the variety of compounds available and the differing efficiencies of various routes of administration. For example, oral administration would be expected to require higher dosages than administration by intravenous injection. Variations in these dosage levels can be adjusted using standard empirical routines for optimization, as is well understood in the art. In certain embodiments, the present invention contemplates a similar dosage for Parkinson's disease. For example, Levodopa (L-DOPA) for Parkinson's is typically given at a rate of 200-400 mg/day and doses up to 1200 mg/day can be used. The term “peripheral” as used herein, means outside of the CNS.

### Exeplification

The invention now being generally described, it will be more readily understood by reference to the following examples, which are included merely for purposes of illustration of certain embodiments and embodiments of the present invention, and are not intended to limit the invention.

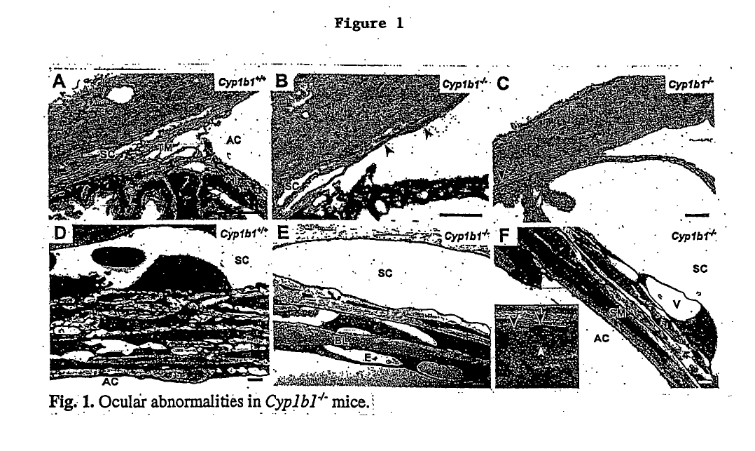

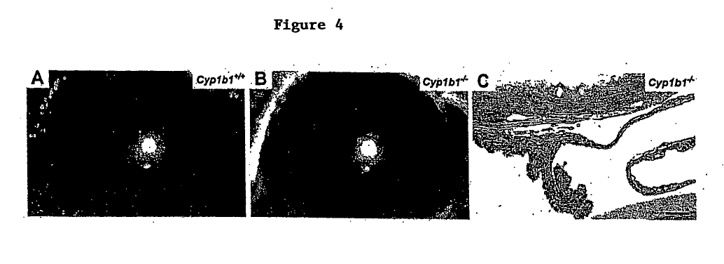

To study ocular developmental abnormalities underlying PCG, Applicants evaluated the consequences of Cyp1b1 deficiency in mice. The development and structure of the trabecular meshwork and Schlemm's canal iridocorneal angle drainage structures are similar in humans and mice (8). Homozygous mutant mice (Cyp1b1^{-/-}) on a mixed 129X1/SvJ×C57BL/6J (B6) background had no gross abnormalities at ages up to 13 months (FIG. 4A and 4B) and their intraocular pressure was indistinguishable from normal littermates (9). However, histological and electron microscopic analyses detected angle abnormalities in all Cyp1b1^{-/-} eyes but not in Cyp1b1^{+/+} eyes (FIG. 1 and FIG. 4C). These defects were only focally present, with much of the angle having normal morphology. Observed defects included small or absent Schlemm's canal, basal lamina extending from the cornea over the trabecular meshwork, fibers resembling smooth muscle at anterior positions in the trabecular meshwork, and attachments of the iris to the trabecular meshwork and peripheral cornea (synechiae). Importantly, these developmental abnormalities resemble those reported in human patients with PCG (10, 11).

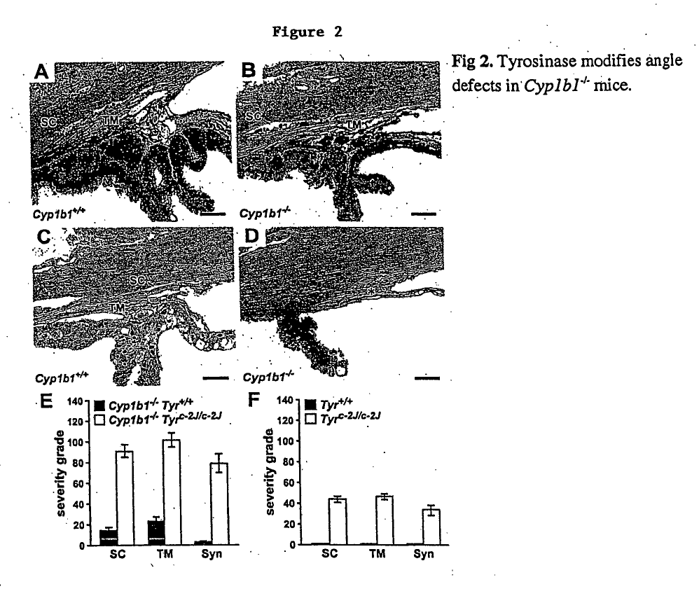

Applicants next tested different mouse strains for strain-specific modifier genes that might suppress or enhance angle abnormalities in these mice. The Cyp1b1 mutation was crossed onto strains 129X1/SvJ, B6, CAST/Ei and MOLC/Rk. No gross defects or elevation in intraocular pressure were present in Cyp1b1^{-/-} mice on any of these backgrounds (9). However, histologic studies provided initial evidence for a modifier gene (tyrosinase, the rate-limiting enzyme in the pigment production pathway). Pigmented Cyp1b1^{-/-} mice of the B6 background had mild and focal angle abnormalities, including small Schlemm's canal and hypoplastic trabecular meshwork (FIG. 2). However, albino (tyrosinase deficient) Cyp1b1^{-/-} mice of the 129X1/SvJ background had more severe and extensive angle abnormalities (9). Examination of the segregating 129X1/SvJ×B6 background, also showed that albino mice lacking tyrosinase were more severely affected than p igmented mice [FIG. 1 and (9)]. Although the albino and pigmented mice of the above strains had many genetic differences, these observations suggested that the presence of tyrosinase protects against developmental abnormalities in Cyp1b1^{-/-} mice.

To test whether the Tyr gene modifies angle abnormalities in Cyp1b1^{-/-} mice, Applicant analyzed pigmented and albino Cyp1b1^{-/-} B6 mice that were genetically uniform except for the presence or absence of the mutant Tyr^{c-2J} allele that arose on this genetic background (12). Applicant assessed the severity of angle abnormalities by analysis of histological sections from different regions around these eyes, and devised a severity grading protocol to allow comparisons between different eyes (12). Three separate angle abnormalities that contribute to glaucoma were analyzed (FIG. 2). Pigmented Cyp1b1^{-/-} mice consistently had mild focal defects whereas albino Cyp1b1^{-/-} mice had severe and more extensive developmental defects (FIG. 2A-D). These differences were consistent and significant for all assessed phenotypes (FIG. 2E; P<0.001 for all).

Applicant also analyzed albino and pigmented B6 mice (Cyp1b1^{+/+}) to determine if the Tyr genotype alters susceptibility of B6 eyes to anterior segment dysgenesis. Albino Cyp1b1^{+/+} mice were found to have mild focal developmental defects whereas pigmented Cyp1b1^{+/+} mice had no observed defects (FIG. 2F), thus confirming the protective effect of tyrosinase activity. Importantly, the severity of abnormalities in albino Cyp1b1^{-/-} mice was greater than accounted for by adding the severity values of pigmented Cyp1b1^{-/-} and albino Cyp1b1^{+/+} eyes (FIG. 2, E and F). Together, these results demonstrate an interaction (exacerbating angle dysgenesis) between Tyr and Cyp1b1 deficiency.

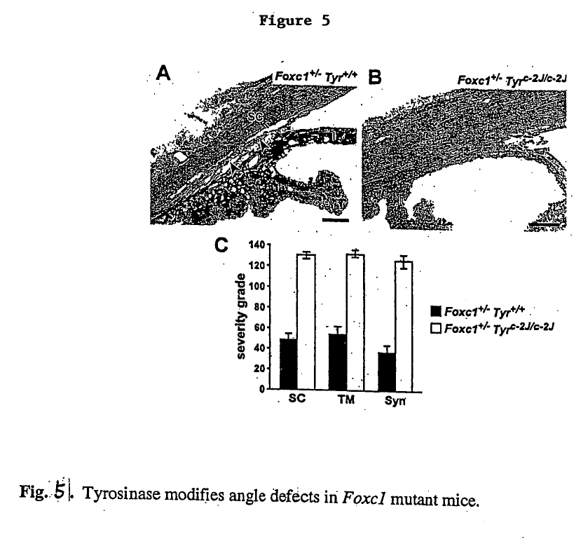

Human PCG can also arise from dominant mutations in the transcription factor gene FOXCi (forkhead box C1, formerly FKHL7) (5). Foxc1^{+/-} mice have anterior segment dysgenesis phenotypes resembling those in human patients (13, 14). To determine if Tyr activity also modifies the phenotype in Foxc1^{+/-} mice, Applicant compared the extent of anterior segment dysgenesis in Foxc1^{+/-} mice that were genetically uniform except for the presence or absence of the Tyr mutation. Tyrosinase deficient Foxc1^{+/-} mice had more severe abnormalities than their pigmented counterparts (B6 background; FIG. 5). Abnormalities in Schlemm's canal and the extent of synechiae were more severe in albino Foxc1^{+/-} mice than accounted for by adding the severity grades for albino Foxc1^{+/+} mice and pigmented Foxc1^{+/-} mice, suggesting that the combined effects of Foxc1 and Tyr mutation are more than additive. Together, our experiments identify a previously unknown anterior segment developmental pathway involving tyrosinase, and show that Tyr mutation modifies the phenotype associated with inheritance of mutant orthologs of two known human PCG genes, Cyp1b1 and Foxc1.

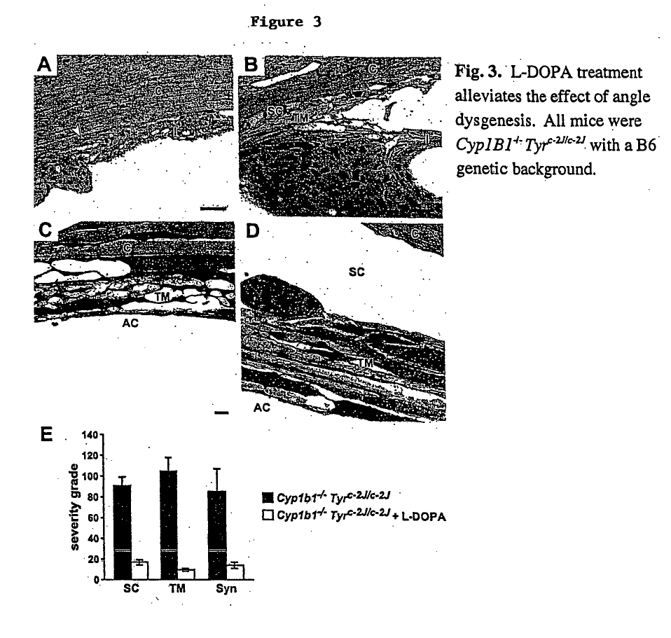

Applicant hypothesized that tyrosinase might affect angle development through modulation of L-DOPA levels. Tyrosinase is a monooxygenase that converts tyrosine to L-DOPA and L-DOPA to dopaquinone. L-DOPA affects development and is the precursor of catecholamines, which are important developmental regulators (15-17). To investigate whether L-DOPA is a critical molecule in angle development, Applicant administered water containing L-DOPA or standard drinking water to Cyp1b1^{-/-} Tyr^{c-2J/c-2J} (albino) double mutants throughout ocular development [their mothers received this water throughout the period of in utero development (12)]. L-DOPA treatment was found to prevent the severe angle dysgenesis present in untreated mice lacking both CYP1B1 and TYR (FIG. 3). This experiment demonstrates that a pathway involving L-DOPA or an L-DOPA metabolite participates in angle formation and that disturbances in this pathway can be treated by L-DOPA administration. Despite the profound rescue in L-DOPA treated albino Cyp1b1^{-/-} mice, mild abnormalities of similar severity to those observed in pigmented Cyp1b1^{-/-} mice remained. Our experiments do not distinguish if these remaining abnormalities reflected an inability of L-DOPA administration to rescue Cyp1b1 phenotypes or if Cyp1b1 phenotypes were rescued but DOPA treatment was not completely effective (possibly due to variability in achieved L-DOPA levels at different angle locations). Importantly, alleviation of ocular defects by L-DOPA suggests that other genes that are unlikely to alter pigmentation are potential candidates to affect angle formation. These include genes that affect L-DOPA levels, affect the metabolism of L-DOPA to other developmentally important molecules or affect the signaling of these molecules in developmental/physiologic pathways.

Anterior segment development occurs by similar processes and is affected by many of the same genes in human and mice (3, 8). That Tyr function protects against angle dysgenesis in both the Cyp1b1^{-/-} and Foxc1⁺^{/-} mouse models raises the possibility that mutations in multiple genes contributing to developmental glaucoma affect L-DOPA levels. L-DOPA levels may be altered in the neural crest cells from which the angle structures and iris stroma derive. Conceivably, mutations in glaucoma genes impact the activity of tyrosine hydroxylase (TH), which produces L-DOPA from tyrosine. Many of the genes that cause anterior segment dysgenesis and/or developmental glaucoma can promote either TH expression or the proliferation of TH expressing neural crest cells during the development of other tissues (18-23). PITX2 and PITX3, for example, induce Th expression by binding to a high affinity regulatory site in the Th gene (21). Additionally, CYP1B1 oxidizes all-trans-retinol to all-trans-retinal, the rate limiting step for retinoic acid biosynthesis (24), and retinoic acid promotes proliferation of a subset of avian neural crest cells that express TH (25). Together, these observations support a model in which a metabolic disturbance involving TH and L-DOPA levels contributes to the anterior segment dysgenesis caused by mutations in various glaucoma genes.

Anterior segment dysgenesis and congenital glaucoma have been reported in a few humans with albinism (26, 27), but this coexistence of phenotypes has typically been considered a coincidence. Our experimental data along with a 1984 report of anterior segment dysgenesis in 7% of 86 studied albinos (27), suggests that tyrosinase may play a role in congenital glaucoma in humans. Abnormalities in genes other than TYR also result in decreased ocular pigmentation (28) and deficiency of at least one of these genes, Tyrpl, decreases tyrosinase stability (29). Our data raise the possibility that these mutations may modify the effects of developmental glaucoma genes by affecting L-DOPA levels. Finally, our findings suggest that L-DOPA may merit investigation as a possible therapy for reducing the incidence of glaucoma in certain high-risk families.

### References for Background of the Invention and Exemplification

1. R. Ritch, M. B. Shields, T. Krupin, The Glaucomas, Clinical Science. R. Ritch, M. B. Shields, T. Krupin, Eds. (Mosby), St. Louis, MO, ed. 2nd, 1996), vol. 2.

2. A. L. Vincent et al., Am J Hum Genet 70, 448 (2002).

3. D. B. Gould, S. W. M. John, Hum Mol Genet 11, 1185 (2002).

4. M. Sarfarazi, I. Stoilov, Eye 14,422 (2000).

5. D. Y. Nishimura et al., Nature Genet. 19, 140 (1998).

6. A. Gencik, A. Gencikova, A. Gerinec, Clin. Genet. 17, 241 (1980).

7. B. A. Bejjani et al., Hum. MoL Genet. 9, 367 (2000).

8. R. S. Smith, A. Zabaleta, O. V. Savinova, S. W: M. John, BMC Dev. Biol. 1, 3 http://www.biomedcentral.com/1471-213X/1/3 (2001).

9. O. V. Savinova, R. S. Smith, S. W. M. John. Unpublished observations.

10. L. Allen, H. M. Burian, A. E. Braley, Arch. Ophthalmol. 53, 783 (1955).

11. E. A. Maumenee, Trans. Am. Ophthalmol. Soc. 56, 507 (1958).

12. Materials and methods are available as supporting material on Science Online.

13. H. K. Hong, J. H. Lass, A. Chakravarti, Hum. Mol. Genet. 8, 625 (1999).

14. R. S. Smith et al., Hum. Mol. Genet. 9, 1021 (2000).

15. M. Ilia, G. Jeffery, J Comp. Neurol. 405, 394 (1999).

16. S. A. Thomas, A. M. Matsumoto, R. D. Palmiter, Nature 374, 643 (1995).

17. Q. Y. Zhou, C. J. Quaife, R. D. Palmiter, Nature 374, 640 (1995).

18. T. L. Dellovade, D. W. Pfaff, M. Schwanzel-Fukuda, J Comp. Neurol. 402, 402 (1998).

19. T. Vitalis et al., J Neurosci. 20, 6501-6516 (2000).

20. P. Cazorla, M. P. Smidt, K. L. O'Malley, J. P. Burbach, J Neurochem. 74, 1829 (2000).

21. M. Lebel, Y. Gauthier, A. Moreau, J. Drouin, J Neurochem. 77,558 (2001).

22. J. E. Varley, G. D. Maxwell, Exp. Neurol. 140, 84 (1996).

23. M. P. Smidt et al., Nat. Neurosci. 3, 337 (2000).

24. H. Chen, W. N. Howald, M. R. Juchau, Drug Metab. Dispos. 28, 315 (2000).

25. J. M. Rockwood, G. D. Maxwell, Exp. Cell Res. 223, 250 (1996).

26. R. A. Catalano, L. B. Nelson, D. B. Schaffer, Ophthalmic Paediatr. Genet. 9, 5 (1988).

27. D. B. van Dorp, J. W. Delleman, D. H. Loewer Sieger, Clin. Genet. 26, 440 (1984).

28. R. E. Boissy, J. J. Nordlund, Pigment Cell Res 10, 12 (1997).

29. T. Kobayashi, G. Imokawa, D. C. Bennett, V. J. Hearing, J Biol Chem 273, 31801 (1998).

### Material and Methods

Animal husbandry and stocks—All experiments were conducted in compliance with the Association for Research in Vision and Ophthalmology statement on the use of animals in ophthalmic and vision research. Mice were housed under previously defined conditions (1). The Jackson Laboratory's routine surveillance program screened for select pathogens. The Cyp1b1^{tm/Gonz} mutation (2) (herein refereed to as Cyp1b1) was crossed to strain 129X1/SvJ for 5 generations, strain C57BL/6J for 8 or more generations, and strains CAST/Ei and MOLC/Rk for 2 generations. Heterozygotes were intercrossed to produce Cyp1b1^{+/+} and Cyp1b1^{-/-} mice. The albino tyrosinase allele that Applicants used was the C57BL/6J Tyr^{c-2J} allele (3). The Foxc1^{Lacz} mutation (4) (herein referred to as Foxc1⁻) was crossed to strain C57BL/6J for 10 or more generations.

L-DOPA treatment—L-DOPA (dihydroxyphenylalanine, 15,431-8, Aldrich Chemical Company, Inc., Milwaukee, Wis.) was supplied to the animals via their drinking water at a concentration of 1 mg/ml (5, 6). L-DOPA was dissolved into acidic water (pH of 3) to avoid oxidation. Animals were either on L-DOPA for their entire gestation or started on L-DOPA at embryonic day E5.5 (prior to eye formation). Eyes were harvested at approximately 4 weeks of age and L-DOPA treatment was continued until the time of harvest. Freshly made L-DOPA water was supplied every three days.

Clinical examinations and intraocular pressure—Applicant examined at least mice of each genotype in age and sex matched groups on each strain background. The mice were analyzed at ages ranging from 3 months to 14 months. Slit-lamp examination and intraocular pressure measurements were performed as previously described (1, 7, 8).

Histologic and electron microscopic analyses—Applicant fixed and processed eyes for plastic sectioning and electron microscopy as previously reported (1, 9). All tissues were harvested from mice at ages after angle formation. For light microscopy, eyes were sagittaly sectioned and up to 56 sections were collected from each of 3 different ocular regions for all eyes. The lens was used as a landmark in collecting sections to ensure that they were collected from comparable regions in all eyes. Collected regions included nasal lens periphery, central lens, and a region halfway between the lens center and the peripheral lens. Sections were stained with hematoxylin and eosin. Due to the potential for artifact in the delicate tissues analyzed abnormalities had to be present in multiple sections from the same region. Nine to twelve eyes were analyzed for each genotype on each genetic background. Applicant devised a grading scheme to allow comparison between different eyes. Applicant separately assessed the severity of defects affecting Schlemm's canal (SC), trabecular meshwork (TM) and creating synechiae. For each phenotype, Applicant graded 6 similarly spaced sections for each ocular region. Applicant graded the severity of abnormalities in both angles of a section. Thus, Applicant graded 36 angles (6 sections×2 angles×3 regions) from different ocular regions for each eye. Applicant designated a grade of 0, 1, 2, 3, or 4 (increasing in severity from 0 to 4, see below) to each phenotype for each angle. For an eye, the total grade could be any value between 0 and 144 (36 angles×4). A total grade of 0 indicated that the angle was normal at all analyzed locations around the eye, while a score of 144 would have indicated that the angle was severely affected at all locations studied. To assess the difference in the severity of angle dysgenesis between the albino and pigmented eyes, Applicant graded approximately 10 eyes from mice of each Cyp1b1 and Foxc1 genotype. Due to the continuous nature of the grade, Applicant present the data as mean± SEM and compared genotypes using two tailed student t-tests. Applicant checked the consistency of the grading method by assessing 8 random eyes on a second occasion without knowledge of the initial grade. The difference in grades between each examination was very small. Eyes of different Cyp1b1 and Foxc1 genotypes were intermixed during grading and the investigator was not aware of their genotype.

Applicant graded Schlemm's canal based on its size (0=normal range, 1=one third to two thirds normal length, 2=small to one third normal length, 3=barely visible, 4=absent). Applicant graded trabecular meshwork based on the morphology and number of trabecular beams and intertrabecular spaces (0=normal with 6 to 8 robust trabecular beams and open intertrabecular spaces, 1=fewer than normal trabecular beams (4 to 6) often with a hypoplastic appearance and open intertrabecular spaces, 2=approximately half normal number of trabecular beams often of hypoplastic appearance with fewer than normal intertrabecular spaces, 3=only 1 to 2 trabecular beams with no visible intertrabecular spaces, TM appeared stalled at an earlier stage of development, 4=no recognizable TM). Applicant graded synechiae based on how far they extended (0=normal, iris joins ciliary body at the iris root and is not attached to the trabecular meshwork, 1=iris is attached to the very posterior TM, 2=iris is attached to three quarters or more of the TM, 3=iris covers entire TM and extends onto peripheral cornea, 4=iris covers TM and extends further onto cornea).

### References for Methods and Materials

1. R. S. Smith et al., Hum. Mol. Genet. 9, 1021-1032 (2000).

2. J. T. Buters et al., Proc. Natl. Acad. Sci. U. S. A. 96, 1977-1982 (1999).

3. N. Le Fur, S. R. Kelsall, B. Mintz, Genomics 37, 245-248 (1996).

4. T. Kume et al., Cell 93, 985-996 (1998).

5. M. Rios et al., J Neurosci 19, 3519-26 (1999).

6. Q. Y. Zhou, C. J. Quaife, R. D. Palmiter, Nature 374, 640-643 (1995).

7. S. W. M. John et al., Invest. Ophthalmol. Vis. Sci. 38, 249-253 (1997).

8. O. V. Savinova et al., BMC Genet 2, 12 (2001).

9. R. S. Smith, S. W. M. John, P. M. Nishina, J. P. Sundberg, Eds., Systematic Evaluation of the Mouse Eye: Anatomy, Pathology and Biomethods (CRC Press, Baco Raton, 2002).

### INCORPORATION BY REFERENCE

All publications and patents mentioned herein are hereby incorporated by reference in their entirety as if each individual publication or patent was specifically and individually indicated to be incorporated by reference.

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

We claim:

1. A method for treating an individual suffering from glaucoma, comprising administering to the individual a therapeutically effective amount of a compound that increases DOPA or DOPA metabolite activities, and/or modulates at least one downstream signaling pathway.

2. The method of claim 1, wherein the compound is selected from the group consisting of: L-DOPA, dopaquinone, dopamine, an L-DOPA derivative, an L-DOPA metabolite, and a catecholamine.

3. The method of claim 1, wherein the compound increases the mono-oxygenation or hydroxylation of tyrosine.

4. The method of claim 3, wherein the compound increases the activity of an enzyme with tyrosine hydroxylase or tyrosine monoxygenase activity.

5. The method of claim 3, wherein the compound increases the expression of an enzyme with tyrosine hydroxylase or tyrosine monoxygenase activity.

6. The method of claim 1, wherein the glaucoma is a developmental glaucoma.

7. The method of claim 1, wherein the onset of glaucoma is in a juvenile or in an adult.

8. The method of claim 1, wherein the compound is formulated with a pharmaceutically acceptable carrier.

9. The method of claim 8, wherein the compound is administered orally, intravitreally, topically, ocularly or parenterally.

10. The method of claim 1, further comprising administering a second glaucoma therapeutic agent selected from the group consisting of: a miotic, epinephrine; a beta-blocker; a carbonic anhydrase inhibitor; an alpha-adrenergic agonist; and a prostaglandin analog.

11. The method of claim 1, further comprising treating the individual with a glaucoma therapy selected from the group consisting of: trabeculoplasty; iridotomy; and cyclophotocoagulation.

12. A method of reducing damage to retinal ganglion cells associated with glaucoma in an individual, comprising administering to the individual a compound that increases DOPA or DOPA metabolite activities, and/or modulates at least one downstream signaling pathway, in an amount sufficient to reduce glaucoma-associated damage to the cells.

13. The method of claim 12, wherein the compound is selected from the group consisting of: L-DOPA, dopaquinone, dopamine, an L-DOPA derivative, an L-DOPA metabolite, and a catecholamine.

14. The method of claim 12, wherein the compound increases the mono-oxygenation or hydroxylation of tyrosine.

15. The method of claim 14, wherein the compound increases the activity of an enzyme with tyrosine hydroxylase or tyrosine monoxygenase activity.

16. The method of claim 14, wherein the compound increases the expression of an enzyme with tyrosine hydroxylase or tyrosine monoxygenase activity.

17. The method of claim 12, wherein the glaucoma is a developmental glaucoma.

18. The method of claim 12, wherein the onset of glaucoma is in a juvenile or in an adult.

19. A method of preventing or reducing the onset of glaucoma in an individual, comprising administering to the individual a therapeutically effective amount of a compound that increases DOPA or DOPA metabolite activities, and/or modulates at least one downstream signaling pathway.

20. The method of claim 19, wherein the compound is selected from the group consisting of: L-DOPA, dopaquinone, dopamine, an L-DOPA derivative, an L-DOPA metabolite, and a catecholamine.

21. The method of claim 19, wherein the compound increases the mono-oxygenation or hydroxylation of tyrosine.

22. The method of claim 21, wherein the compound increases the activity of an enzyme with tyrosine hydroxylase or tyrosine monoxygenase activity.

23. The method of claim 21, wherein the compound increases the expression of an enzyme with tyrosine hydroxylase or tyrosine monoxygenase activity.

24. A method of predicting glaucoma in an individual, comprising measuring the level of L-DOPA in the aqueous humor of the individual, wherein a reduced level of L-DOPA in the aqueous humor is indicative of increased likelihood that the individual will develop glaucoma.

25. A method of diagnosing glaucoma in an individual, comprising measuring the level of L-DOPA in the aqueous humor of the individual, wherein a reduced level of L-DOPA in the aqueous humor is indicative of glaucoma in the individual.

26. A method of predicting glaucoma in an individual, comprising measuring the function of an enzyme with tyrosine hydroxylase or tyrosine monoxygenase activity in the individual, wherein a reduced function of the enzyme is indicative of increased likelihood that the individual will develop glaucoma.

27. The method of claim 26, wherein the function includes activity and expression level of the enzyme.

28. A method of diagnosing glaucoma in an individual, comprising measuring the function of an enzyme with tyrosine hydroxylase or tyrosine monoxygenase activity in the individual, wherein a reduced function of the enzyme is indicative of glaucoma.

29. The method of claim 28, wherein the function includes activity and expression level of the enzyme.

30. A method for treating an individual with ocular manifestation associated with albinism or with ocular albinism, comprising administering to the individual a therapeutically effective amount of a compound that increases DOPA or DOPA metabolite activities, and/or modulates at least one downstream signaling pathway.

31. The method of claim 30, wherein the compound is selected from the group consisting of: L-DOPA, dopaquinone, dopamine, an L-DOPA derivative, an L-DOPA metabolite, and a catecholamine.

32. The method of claim 30, wherein the compound increases the mono-oxygenation or hydroxylation of tyrosine.

33. The method of claim 32, wherein the compound increases the activity of an enzyme with tyrosine hydroxylase or tyrosine monoxygenase activity.

34. The method of claim 32, wherein the compound increases the expression of an enzyme with tyrosine hydroxylase or tyrosine monoxygenase activity.

35. A pharmaceutical composition comprising: (a) a first compound that increases DOPA or DOPA metabolite activities, and/or modulates at least one downstream signaling pathway, and (b) a second compound selected from the group consisting of: a miotic, epinephrine; a beta-blocker; a carbonic anhydrase inhibitor; an alpha-adrenergic agonist; and a prostaglandin analog.

36. The pharmaceutical composition of claim 35, wherein the first compound is selected from the group consisting of: L-DOPA, dopaquinone, dopamine, an L-DOPA derivative, an L-DOPA metabolite, and a catecholamine.

37. The pharmaceutical composition of claim 35, wherein the first compound increases the mono-oxygenation or hydroxylation of tyrosine.

38. The pharmaceutical composition of claim 37, wherein the first compound increases the activity of an enzyme with tyrosine hydroxylase or tyrosine monoxygenase activity.

39. The pharmaceutical composition of claim 37, wherein the first compound increases the expression of an enzyme with tyrosine hydroxylase or tyrosine monoxygenase activity.
